(19) United States
(12) Patent Application Publication
(10) Pub. No.: US 2011/0082099 A1
(43) Pub. Date: Apr. 7, 2011

(54) Methods and Compositions for the Treatment of Cystic Fibrosis and Related Illnesses

(76) Inventor: Melanie Childers, Baycliff, TX (US)

(21) Appl. No.: 12/815,392

(22) Filed: Jun. 14, 2010

Related U.S. Application Data
(62) Division of application No. 11/411,250, filed on Apr. 26, 2006, now abandoned.

(51) Int. Cl. A61K 31/7036; A61K 31/26; A61K 31/546; A61K 31/496; A61K 31/397; A61P 9/00; A61P 31/00; A61P 43/00

(52) U.S. Cl. 514/40; 514/514; 514/515; 514/203; 514/252.04; 514/210.15

(57) ABSTRACT

The present invention relates to methods and compositions to treat subjects having cystic fibrosis. These compositions comprise the class of isothiocyanates. Isothiocyanates, absorbed by a cell are conjugated with glutathione GSH by glutathione-s-tranferase (GST). The conjugates are substrates of the multi-drug resistance associated (MRP)/multi-drug resistance (MDR) proteins. These proteins are functionally redundant to the cystic fibrosis transmembrane conductance regulator (CFTR), allowing for the substrate conjugates to be exported from the cell. The export of GSH conjugates restores intracellular and extracellular levels of GSH to normal levels. Normalizing both extracellular and intracellular GSH via the increased conjugation of isothiocyanates with GSH, and subsequent export, can significantly rectify numerous enzymatic processes and correct the pathologies that are typical of patients suffering from cystic fibrosis.

### CROSS REFERENCE TO RELATED APPLICATION

This application claims the benefit of U.S. provisional application 60/675,198 filed on Apr. 27, 2005, and U.S. patent application Ser. No. 11/411,250, publication no 20060258599, filed Nov. 16, 2006.

The present invention relates to the field of treating cystic fibrosis by administering a pharmaceutically effective amount of a composition comprising one or members of a class of compound known as isothiocyanate, resulting in normalization of intracellular and extracellular levels of glutathione (“GSH”) and the correction of numerous pathologies that are known of this disease.

### BACKGROUND OF INVENTION

Cystic fibrosis (CF) is a genetic disease affecting approximately 30,000 children and adults in the United States. At the root of this condition is a defective gene that prevents cells from producing functional Cystic Fibrosis Transmembrane Conductance Regulator proteins (CFTRs). The missing or non-functional CFTRs undermine the body's immune system, cause hyperinflammation and cause the body to produce abnormally thick, sticky mucus that clogs the small airways of the lungs and leads to life-threatening lung infections. These thick secretions obstruct other exocrine glands, including the pancreas, preventing digestive enzymes from reaching the intestines to help break down and absorb food.

This invention identifies methods, compounds, and classes of compounds that work at the cellular level to treat cystic fibrosis.

Glutathione (GSH) is a ubiquitous tripeptide found intracellularly and extracellularly in plants and animals. Each glutathione molecule is composed of an N-terminal glutamine linked to a cysteinyl group, through a .gamma.-carbamyl linkage, followed by a glycine. While GSH is resistant to proteolysis, each GSH molecule harbors a thiol group that is extremely active in reduction reactions. GSH reduces many oxidants, for example, hydrogen peroxide, which is toxic at high levels. GSH is vital to biological systems and regulates many signaling pathways via its reducing capabilities.

In the extracellular lung fluid (ELF) of the lung, GSH combats extracellular oxidants. In the normal lung, when oxidant stress increases, such as in asthmatics or smokers, extracellular levels of GSH are concomitantly increased. Lung epithelial cells are partly responsible for this increase; in vitro experiments reveal that they increase intracellular GSH production when exposed to extracellular reactive oxidant species (ROSs). The CFTR protein then transports GSH out of the cell whereby the GSH neutralizes the ROSs. Within the cell, GSH is often exposed to a thiol-disulfide exchange reaction that is catalyzed by a thiol-transferase. This is a reversible reaction wherein the equilibrium is determined by the redox state of the cell. This redox state is dependent upon the overall cellular concentrations of GSH (thiol) and GSSG (disulfide). The thiol-disulfide equilibrium within the cell regulates a diverse number of metabolic processes, including enzyme activity, transport activity, gene expression by redox-sensitive trans-activating factors, and immune response.

Studies show that a normal CFTR transports GSH out of the cell. See, for example, Linsdell and Hanrahan, AM J Physiol. 1998 July; 275 (1 Pt 1):C323-6. In vitro studies show that cystic fibrosis (CF) lung epithelial cells efflux less GSH into the extracellular fluid than cells that have a normal CFTR. Adults with cystic fibrosis show a two-fold reduction in their plasma GSH levels and a 10- to 20-fold reduction in lung extracellular GSH levels compared to normal individuals. The synthesis of GSH in cells of CF patients, however, is not decreased. GSH is normally present at 2-10 mM inside cells. See, for example, Gao et al., Am J Physiol. 1999 July; 277 (1 Pt 1):L113-8. Most of these cells derive their GSH from the de novo synthesis of GSH. Normal human epithelial lung lining fluid (ELF) contains .about.400 .mu.M GSH. This is .mu.140-fold higher than that in the plasma. See, for example, Cantin et al., J. Appl. Physiol. 63:152-157, 1987. It is here, in the extracellular environment, that GSH functions as a neutralizer of free radicals generated by lipid peroxidation and hypochlorous acid produced by neutrophils during inflammation. GSH content is significantly decreased in the lung lavage fluid from adult CF patients. See, for example, Roum et al., J. Appl. Physiol. 75: 2419-2424, 1993. Therefore, a defective or missing CFTR results in crippling GSH transport, even though GSH production is not affected. The result is a deficiency of extracellular GSH and supraphysiological levels of intracellular GSH.

The present invention centers on the transport of GSH out of CF cells. The family of multidrug resistance proteins, collectively known as the MDR and MRP, are responsible for multidrug resistance and the transport of chemotoxic drugs out of the intracellular space. Because these proteins are functionally similar to the CFTR, the MDR and MRP can provide some of the functionality that the CFTR normally provides. It is well known by those who are trained in the art that cellular MRP and MDR proteins export GSH conjugates when the cells undergo a process in which the liver uses one of two major enzyme pathways to change a toxic substance, such as an anticancer drug, into a less toxic substance that is easier for the body to excrete (phase II detoxification). For example, see Zaman et al., Proc. Natl. Acad. Sci. USA; Vol. 92, pp. 7690-7694, August 1995. Zaman showed that MRP increases the export of glutathione from MRP-transfected lung cancer cell lines. The results of Zaman provided strong support for MRP functioning as a glutathione S-conjugate carrier. In phase II detoxification, liver cells add a substance (such as cysteine, glycine, or a sulfur molecule) to a toxic chemical or drug, to make it less harmful. Furthermore, it has been shown that sulforaphane is transported by MRPs as a glutathione conjugate or adduct. See, for example, Yeusheng and Callaway, Biochem. J. (2002) 364, 301-307. See Zhang, Carcinogenesis. 2000 June; 21(6): 1175-82; wherein two isothiocyanates (sulforaphane and benzyl-ITC) were shown to be conjugated to reduced glutathione (GSH) within Hepa 1c1c7 murine hepatoma cells.

The MDR/MRP proteins serve well as an alternate exportation system for the surplus of GSH, as long as the GSH is carried out in the form of a conjugate with another molecule. Further, by causing the expression of the MDR/MRP proteins with isothiocyanate, or isothiocyanate analogs and derivative compounds, the MRP/MDR will transport the conjugates out of the CF cell and thereby normalize the intracellular and extracellular concentrations of GSH. See, for example, Callaway et al., Cancer Lett. 2004, Feb. 10; 204 (1):23-31; Hu et al., J. Pharm Sci., 2004 July; 93(7): 1901-11; and Zhang and Callaway, Biochem. J., 2002 May 15; 364 (Pt 1):301-7. The family of isothiocyanates, and isothiocyanate analog and derivative compounds, described in this invention cause the expression and activation of these proteins and is central to the therapy described in herein.

Metallothionein (MT) is a small, cysteine-rich metal-binding protein found in the cytoplasm of many eukaryotes. MT contains between 60 and 68 amino acids, of which 20 are highly-conserved cysteines. MT chelates and delivers metals, including zinc and copper, to enzymes that require these metals as cofactors in enzymatic processes. Within the cell, MT is redox sensitive. In a reducing environment, MT chelates and holds, for example, zinc. In an oxidizing environment, MT releases zinc, for example. In a CF cell, high intracellular levels of GSH create a reducing environment. Thus, in a CF cell, MT does not release the metals numerous enzymatic processes need. More specifically, excess GSH oxidizes a higher percentage of the H.sub.2O.sub.2 which results in zinc remaining bound to MT. Zinc is therefore unavailable to enzymatic processes. The increased intracellular GSH mediates the binding of zinc to MT. In a normal cell, thiocyanate (of which isothiocyanate is a naturally occurring isomer) conjugates with GSH and the conjugates are transported out of the cell through the CFTR protein. Furthermore, zinc inhibits phospholipase A2 (PLA2) release of arachidonic acid.

GSH conjugates are not transported out of a CF cell. In a CF cell, because free zinc is not available, PLA2 promotes the release of AA from the cell membrane. The end result of the foregoing processes in a normal cell is that the exported thiocyanate is converted to hypothiocyanate (OSCN) via the action of myeloperoxidase and H.sub.2O.sub.2. Because the CFTR protein is unable to transport any GSH-thiocyanate conjugates to the extracellular space, extracellular thiocyanate cannot effectively neutralize H.sub.2O.sub.2. See FIG. 1.

As described above, the lack of zinc cofactors causes enzymatic processes to be abnormal. For example, thymulin is a hormone that modulates cell mediated immunity and is activated only when it is bound to zinc. Because excess intracellular GSH prevents the release of zinc from MT, there is an inadequate supply of free zinc to activate adequate levels of the hormone. Studies show that people with CF have normal levels of thymulin but that much of it is inactive. Therefore, increased levels of intracellular GSH lead to compromised immune functioning.

The lack of transport of thiocyanate adducts or conjugates may explain why CF patients have reduced bacterial clearance. CF patients with severe pulmonary damage have been shown to have significantly lower levels of thiocyanate than those with moderate disease. See, for example, Weuffen et al., Padiatr. Grenzgeb. 1991; 30 (3): 205-10; and Ratner, Am. J. Respir. Cell. Mol. Biol. 2000 June: 22 (6): 642-4.

Unfortunately, the current treatment protocols for CF have not changed much in the last ten years. The current standard therapies include replacement digestive enzymes, mucolytics (Pulmozyme), anti-inflammatory medications, antibiotics, and chest physiotherapy. The advent of these therapies, along with a high calorie diet has increased the average lifespan of patients with CF, but still these patents rarely live past the mid thirties. Currently, there are drugs that are in the pipeline for the treatment of CF, which include elastase inhibitors, and antibiotics. Furthermore, there is a very large amount of effort to bring gene therapy to these patients. Many routes of delivery have been tried with gene therapy, which include viral vectors and liposomal aerosols, however, it continues to be very difficult to effectively deliver a gene to the lungs without triggering the immune response and causing further lung pathology.

With regard to use of aerosolized forms of thiocyanate compounds for treating CF, U.S. Pat. No. 6,702,998 to Conner describes the direct application of thiocyanate to airway mucosal surfaces.

### SUMMARY OF THE INVENTION

This invention uses pharmaceutically effective doses of isothiocyanates to effectively treat subjects suffering from cystic fibrosis. Furthermore, given the numerous consequences of extracellular GSH deficiency and excess intracellular GSH, some herein articulated, an effective treatment for cystic fibrosis subjects will correct cellular GSH imbalances.

Although isothiocyanates have been known to be an agent that is useful as an anticarcinogen, it can now be disclosed that this drug is surprisingly efficacious in the treatment of cystic fibrosis.

The present invention relates to a method for correcting the imbalance of fatty acids often exhibited in subjects with CF. The present invention further relates to correcting GSH imbalances that occur as a result of the mutated CFTR protein. The present invention further provides a method of treatment to restore GSH levels comprising administering to subjects in need thereof, a therapeutic amount of isothiocyanate(s) or derivative thereof sufficient to increase extracellular glutathione levels and/or decrease oxidative stress and monitoring restoration by measuring the level of glutathione in blood as needed. Such therapeutic amounts can be determined by measurement or by a physician.

An embodiment of the present invention comprises orally administering a therapeutically effective amount of one or more isothiocyanates, or one or more derivatives or analogs thereof; wherein intracellular expression of MDR and/or MRP proteins is induced; wherein the isothiocyanate, or derivative of analog thereof is intracellularly conjugated to GSH; and wherein the GSH conjugate is exported to the extracellular milieu. In an alternative embodiment, the oral administration of a therapeutically effective amount may be accompanied by the administration of one or more antibiotics. It is preferred that the oral administration of a therapeutically effective amount of isothiocyanates, or a derivative or analog thereof, results in the digestion and absorption of the isothiocyanates, or derivatives or analogs thereof, from the gastrointestinal tract into the cardiovascular and lymphatic systems for distribution to cells.

In another embodiment of the present invention, a therapeutically effective amount of isothiocyanate(s) is provided alone or in combination with a therapeutically effective amount of a therapeutic agent, and one or more pharmaceutically acceptable carriers, excipients, or diluents, wherein the therapeutic agent complements the action or activity of the isothiocyanate. Such therapeutic agents include, but are not limited to, antibiotics such as astreonam, ceftazidime, tobramycin, and ciprofloxacin.

### BRIEF DESCRIPTION OF THE DRAWINGS

FIG. 1: Comparison of the resultant intracellular mechanisms due to (A) a properly functioning CFTR protein (normal cell) and (B) a CFTR protein unable to transport GSH-isothiocyanate conjugates (CF cell).

### DETAILED DESCRIPTION OF THE INVENTION

As used herein, the terms “subject” or “patient” are used interchangeably are used to mean any animal, preferably a mammal, including humans and non-human primates. In one embodiment of the invention, the subject having cystic fibrosis, or a carrier thereof, suffers from fatty acid imbalance(s).

An embodiment of the present invention comprises orally administering a therapeutically effective amount of one or more isothiocyanates, or derivatives or analogs thereof; wherein intracellular expression of MDR and/or MRP proteins is induced; wherein the isothiocyanate, or derivative of analog thereof is intracellularly conjugated to GSH; and wherein the GSH conjugate is exported to the extracellular milieu. In an alternative embodiment, the oral administration of a therapeutically effective amount may be accompanied by the administration of one or more antibiotics. It is preferred that the oral administration of a therapeutically effective amount of isothiocyanates, or a derivative or analog thereof, results in the digestion and absorption of the isothiocyanates, or derivatives or analogs thereof, from the gastrointestinal tract into the cardiovascular and lymphatic systems for distribution to cells.

In another embodiment of the present invention, a therapeutically effective amount of isothiocyanate(s) is provided alone or in combination with a therapeutically effective amount of a therapeutic agent, and one or more pharmaceutically acceptable carriers, excipients, or diluents, wherein the therapeutic agent complements the compound(s) that restore or increase extracellular glutathione levels and restore or decrease intracellular glutathione levels.

In another embodiment, the methods of the current invention may further comprise administering an antibiotic, an antibiotic regimen or another drug to the subject. Antibiotics for use in combination with the compositions of the present invention include, but are not limited to, astreonam, ceftazidime, tobramycin, and ciprofloxacin.

It is contemplated that the antibiotic may be inhaled for the treatment of infections in the lungs associated with CF. As an alternative to swallowing the antibiotic as a pill or injecting it intravenously or intramuscularly, inhalation will deliver the drug specifically to the site of infection where it can directly treat the associated infection(s).

Isothiocyanates are compounds containing the isothiocyanate (−NCS—) moiety and are readily identifiable. Isothiocyanates are isomers of thiocyanate and can induce phase II detoxification using MDR/MRP proteins. Isothiocyanates, such as benzyl isothiocyanate (BITC), increase reactive oxygen intermediates (ROI) inside the cell. ROI's increase the expression of glutathione-S-transferase (GST). GSTs catalyze the conjugation of reactive chemicals with GSH. These reactive chemicals include isothiocyanates and thiocyanates. For example, see Kirlin et al., Journal of Nutrition. 1999; 129: 1827-1835; wherein HT29 cells cultures exposed to benzyl isothiocyanate (BITC) resulted in a statistically significant increase in glutathione S-transferase activity. 5 .mu.mol/L of BITC resulted in .about.0.51 .mu.mol min.sup.-1 mg proteins.sup.-1 of glutathione S-transferase activity. 25 .mu.mol/L of BITC resulted in .about.0.74 .mu.mol min.sup.-1 mg protein.sup.-1 of glutathione S-transferase activity.

Therefore, when cells absorb isothiocyanates, for example, GST conjugates them with GSH. The conjugates, as substrates of the MRP and MDR proteins, are subsequently transported out of the cell. With the sufficient transport of GSH out of the cell, intracellular and extracellular GSH levels are normalized. The treatments proposed in this invention rely upon the administration of one or more isothiocyanates and, in other embodiments, associated treatments that maximize the effectiveness of isothiocyanates.

Isothiocyanates can be purchased from laboratories, or purified from plants, seeds or plant extracts by methods well known in the art. Plants having high levels of isothiocyanates include, but are not limited to, Brassicaceae (Cruciferae), Moringaceae and Resedaceae, which collectively included, but are not limited to, broccoli, broccoli, sprouts, Brussels sprouts, cabbage, cauliflower, cauliflower sprouts, daikon, horseradish, kale, mustard seed, radish, wasabi, horseradish tree (Moringa oleifera), cabbage tree (M. stenopetala), mignonette (Reseda oderata), dyer's rocket (R. luteola) and papaya seeds. Moreover, these cruciferous plants contain high levels of isothiocyanates which occur naturally. Alternatively, plants may be bred to contain high levels of isothiocyanates. Thus, as contemplated by the present invention, food products may be supplemented with a composition or agent comprising isothiocyanates, thiocyanates, analogs thereof, or derivatives thereof. The supplements may be isolated from plants, for example, those described above.

Breeding techniques, have allowed for the production of plants which have high levels of isothiocyanates. Some Brassica (Crucifer) breeding programs are directed to increasing isothiocyanate production levels. In addition, these same breeding programs can include the identification and selection of cultivars that have high levels of isothiocyanates. Different strategies for the crossing, selection, and breeding of new cultivars of Brassicaceae (Cruciferae) are well known.

In addition, these same breeding programs can include the identification and selection of cultivars that have high levels of isothiocyanates. Strategies for the crossing, selection, and breeding of new cultivars of Brassicaceae (Cruciferae) are well known. (Brassica Crops and Wild Allies: Biology & Breeding; S. Tsunoda et al. (eds), Japan Scientific Societies Press, Tokyo pp. 354 (1980); Biology of Brassica Coenospecies; C. Gomez-Campo (ed), Elsevier, Amsterdam p. 489 (1999)). Progeny plants are screened for high levels of isothiocyanates produced at specific plant developmental stages. Plants carrying the trait of interest are identified and the characteristic intensified or combined with other important agronomic characteristics using breeding techniques well known in the art of plant breeding.

Additionally, plants herbal homeopathic preparations, medications or any substance that contain glucorifin as a precursor to the production of the entire class of isothiocyanate is included. The conversion of glucorforin to isothiocyanate or sulphoraphane (an isothiocyanate) in the digestive tract is well known.

Sulforaphane and its analogs are examples of isothiocyanates. The description and preparation of isothiocyanate analogs is described in U.S. Reissue Pat. No. 36,784, and is hereby incorporated by reference in its entirety. In a preferred embodiment, the sulforaphane analogs used in the present invention include 6-isothiocyanato-2-hexanone, exo-2-acetyl-6-isothiocyanatonorbornanae, exo-2-isothiocyanato-6-methylsulfonyinorbornane, 6 isothiocyanato-2-hexanol, 1-isothiocyanato-4-demethylphosphonylbutane, exo-2-(1′-hydroxyethyl_)-5-isothiocyanatonorbornane, exo-2-acetyl-5-isothiocyanatonorbornane, 1-isothiocyanato-5-methylsulfonylpentane, cis-3-(methylsulfonyl)cyclohexylmethylisothiocyanate and trans-3 (methylsulfonyl)cyclohexylmethylisothiocyanate. Isothiocyanates include, but are not limited to, benzyl isothiocyanate (BITC), sulforaphane, sulforaphene, erysolin, erucin, iberin, alyssin, berteroin, iberverin, cherirolin, 5-methylsufinylpentyl isothiocyanate, 6-methylsulfinylhexyl isothiocyanate, 7-methylsulfinylheptyl isothiocyanate, 8-ethylsulfinyloctyl isothiocyanate, 9-methylsulfinylnonyl isothiocyanate, 10-methylsulfinyldecyl isothiocyanate, phenylethyl isothiocyanate 4-(.gamma.-L-rhamnopyranosyloxy) benzyl isothiocyanate, 3-(.gamma.-L-rhamnopyranosyloxy) benzyl isothiocyanate, 4-(4′-O-acetyl-.alpha.-L-rhamnopyranosyloxy) benzyl isothiocyanate or a derivatives, Phenethyl ITC, Phenyl-ITC, 4-Phenylbutyl-ITC, 6-Phenylhexyl-ITC, 5-Phenylpentyl-ITC, 3-Phenylpropyl-ITC, Propyl-ITC, Methyl ITC, 2-Hexyl isothiocyanate, 1-Hexyl isothiocyanate, Ethyl ITC, 2,2-diphenylethyl ITC, 1,2-diphenylethyl ITC, 1-Dodecyl isothiocyanate, Benzyl ITC, and Allyl ITC. Other isothiocyantes also include, but are not limited to, conjugates of isothiocyanates, which include, among others, glutathione-, cysteinylglycine-, cysteinyl-, and N-acetylcysteine-conjugates. It is contemplated that one or more isothiocyanate(s), or analogs thereof, is used in the treatments identified herein.

For administration to subjects, in reference to the methods of treatment of the present invention, a variety of conventional routes may be used including oral, parenteral (e.g., intravenous, intramuscular or subcutaneous), buccal, anal and topical. In general, the compounds of the invention (hereinafter also known as the active compounds) will be administered at proper pharmaceutical dosages that will be determined, based on patient's weight. Preferably the active compound will be administered orally or could be delivered parenterally. However, some variation in dosage will necessarily occur depending on the condition of the subject being treated. The person responsible for administration will, in any event, determine the appropriate dose for the individual subject.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelation and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration in accordance with the present invention, a sterile injectable solution of the active ingredient can be prepared. Solutions of a therapeutic compound of the present invention in either sesame, olive, MCT, or peanut oil or in aqueous propylene glycol may be employed. The aqueous solutions should be suitably adjusted and buffered, preferably at a pH of greater than 8, if necessary and the liquid diluent first rendered isotonic. These aqueous solutions are suitable intravenous injection purposes. The oily solutions are suitable for intra-articular, intramuscular and. subcutaneous injection purposes. The preparation of all these solutions under sterile conditions is readily accomplished by standard pharmaceutical techniques well known to those skilled in the art.

For the methods of the present invention, the active compounds herein disclosed may also be formulated in rectal compositions such as suppositories or retention enemas, e.g., containing conventional suppository bases such as cocoa butter or other glycerides. For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflators may be formulated containing a powder mix of a compound of the invention and a suitable powder base such as lactose or starch.

Dosing can be adjusted to achieve regular and consistent blood levels, as measured by HPLC. In one embodiment, regular and consistent blood levels of isothiocyanate will be on the order of 50 to 1000 ng/ml. In a preferred embodiment, regular and consistent blood levels of isothiocyanate are 50 to 100 ng/ml. In still another preferred embodiment, regular and consistent blood levels of isothiocyanate are 100 to 200 ng/ml. In still another preferred embodiment, regular and consistent blood levels of isothiocyanate are 200 to 300 ng/ml. In still another preferred embodiment, regular and consistent blood levels of isothiocyanate are 300 to 400 ng/ml. In still another preferred embodiment, regular and consistent blood levels of isothiocyanate are 400 to 500 ng/ml. In still another preferred embodiment, regular and consistent blood levels of isothiocyanate are 500 to 600 ng/ml. In still another preferred embodiment, regular and consistent blood levels of isothiocyanate are 600 to 700 ng/ml. In still another preferred embodiment, regular and consistent blood levels of isothiocyanate are 800 to 900 ng/ml. In still another preferred embodiment, regular and consistent blood levels of isothiocyanate are 900 to 1000 ng/ml. In still another preferred embodiment, regular and consistent blood levels of isothiocyanate are 10 to 50 ng/ml. In still another preferred embodiment, regular and consistent blood levels of isothiocyanate are 5 to 40 ng/ml.

The following Example serves only to illustrate the invention, and should not be construed, in any way, to limit the invention.

### EXAMPLES

### Example 1

Individual study based on 10 mg consumption of BITC.

Fatty acids are important in regulating a variety of biologic functions, including inflammatory responses. It has been shown that patients with CF have altered levels of plasma fatty acids. Freedman et al., have demonstrated that arachidonic acid levels are increased and docosahexaenoic acid levels are decreased in affected tissues from cystic fibrosis-knockout mice. See abstract from Freedman et al., N Engl J Med. 2004 Feb. 5; 350 (6):560-9. Furthermore, tissue samples from 38 CF individuals were examined for any fatty acid imbalance. The results indicated abnormally high levels of arachidonic acid and abnormally low levels of docosahexaenoic acid. See Freedman et al., N Engl J Med. 2004 Feb. 5; 350 (6):560-9. The same study also revealed that obligate heterozygotes had fatty acid levels intermediate between those of the CF patients and those of unaffected control subjects. See Freedman et al., N Engl J Med. 2004 Feb. 5; 350 (6):560-9.

An isothiocyanate of 10 mg was taken orally per day for 10 days by an individual who is a carrier of the CF gene. Blood tests were performed before and after the 10-day study to determine the fatty acid levels before and after administration of the isothiocyanate. Table 1 shows the fatty acid profile results of those two blood tests. TABLE-US-00001 TABLE 1+10% Baseline days Change EPA 70 83 18.57 Arachidonic Acid 847 819-3.31 Mead Acid 25 25 0 h-g-Linoleic Acid 243 219 -9.88 Arachidic Acid 56 51-8.93 DHA C22:6W3 248 338 36.29 DPA C22:6W6 23 21-8.7 DPA C22 6W3 77 73-5.19 DTA C22 4W6T 31 33 6.45 Docosanoic Acid C22:1 3 3 0 Docosanoic Acid C22:0 60.2 73.8 22.59 Nervonic Acid 81 79-2.47 Tetracosanoic Acid 44.3 55.2 24.6 Hexacosanoic Acid C24:0 0.3 0.4 33.33 Hexacosanoic Acid C28:1 0.48 0.62 29.17 Priatanic Acid 0.09 0.1 11.11 Phytanic Acid 1.33 1.58 18.8 Tiete Tebrana Ratio 0.031 0.031 0 Total Saturated 3.5 3.5 0 Total Monounsaturated 2.5 2.8 12 Total Polyunsaturated 4.6 4.8 4.35 Total w3 0.5 0.6 20 Total w61 4.1 4.2 2.44 Total Fatty Acids 10.7 10.9 1.87

Of particular note are the dramatic increases in EPA, DHA, Docosanooic Acid, Tetracosanoic Acid, Hexacosanoic Acid, and Phytanic Acid. DHA is characteristically very low in people with CF. Furthermore, in view of the foregoing described results, indicating abnormally high levels of arachidonic acid and abnormally low levels of docosahexaenoic acid in tissue samples from 38 CF patients, the present results clearly illustrate the remedial effect of isothiocyanates in people with CF (−3.37% change for Arachidonic Acid and a +36.29% change for DHA, see Table I).

It is claimed:

1. A method of treating a subject having cystic fibrosis, the method comprising administering a pharmaceutically effective amount of one or more of an isothiocyanate, an analog thereof, or a derivative thereof, to the subject.

2. The method of claim 1 wherein the one or more isothiocyanate(s), or analog thereof, is selected from a group consisting of include benzylisothiocyanate (BITC), thiocyanate, sulforaphane, sulforaphene, erysolin, erucin, iberin, alyssin, berteroin, iberverin, cherirolin, phenylethylisothiocyanate 4-(.gamma.-L-rhamnopyranosyloxy) benzyl isothiocyanate, 3-(.gamma.-L-rhamnopyranosyloxy) benzyl isothiocyanate, 4-(4′-O-acetyl-.alpha.-L-rhamnopyranosyloxy) benzyl isothiocyanate or a derivatives, Phenethyl ITC, Phenyl-ITC, 4-Phenylbutyl-ITC, 6-Phenylhexyl-ITC, 5-Phenylpentyl-ITC, 3-Phenylpropyl-ITC, Propyl-ITC, 2-Hexyl isothiocyanate, 1-Hexyl isothiocyanate, Ethyl ITC, 2,2-diphenylethyl ITC, 1,2-diphenylethyl ITC, 1-Dodecyl isothiocyanate, Benzyl ITC, and Allyl ITC.

3. The method of claim 1 wherein the composition is a food supplement, a dietary supplement or a food additive.

4. The method of claim 1 wherein the composition is a pharmaceutical composition.

5. The method of claim 4, wherein the pharmaceutical composition is administered orally.

6. The method of claim 1, further comprising administering one or more antibiotics to the subject.

7. The method of claim 6, wherein the one or more antibiotics is selected from the group consisting of astreonam, ceftazidime, tobramycin, and ciprofloxacin.

8. The method of claim 7, wherein the one or more antibiotics is inhaled.

9. The method of claim 2, wherein the isothiocyanate is benzylisothiocyanate (BITC).

10. The method of claim 9, wherein the benzyl isothiocyanate is administered as a 10 mg dose per day.

11. The method of claim 5, wherein the oral administration results in the digestion and absorption of the isothiocyanates, or derivatives or analogs thereof, from the gastrointestinal tract into the cardiovascular and lymphatic systems for distribution to cells.

12. The method of claim 1, wherein the pharmaceutically effective amount of one or more of an isothiocyanate, an analog thereof, or a derivative thereof results in a regular and consistent blood level of isothiocyanate on the order of 50 to 1000 ng/ml.
